(19) United States
(12) Patent Application Publication
(10) Pub. No.: US 2014/0368425 A1
(43) Pub. Date: Dec. 18, 2014

(54) ADJUSTING A TRANSPARENT DISPLAY WITH AN IMAGE CAPTURING DEVICE

(71) Applicant: Wes A. Nagara, Commerce Township, MI (US)

(72) Inventor: Wes A. Nagara, Commerce Township, MI (US)

(21) Appl. No.: 14/275,220

(22) Filed: May 12, 2014

Related U.S. Application Data
(60) Provisional application No. 61/834,219, filed on Jun. 12, 2013.

(51) Int. Cl. G06F 3/00

(52) U.S. Cl. CPC G06F 3/005
USPC 345/156

(57) ABSTRACT

A system and a method for adjusting a transparent display with an image capturing device are provided. The system includes a command module to instigate a monitoring of the multi-layer transparent display with the image capturing device; an image receiving module to receive a first image from the image capturing device and a second image from the image capturing device after a predetermined interval; an image processing module to process the received first image and the second image; and a display driving module to re-render the multi-layer transparent display based on the processing of the received first image and the second image.

### CROSS REFERENCE TO RELATED APPLICATIONS

This U.S. patent application claims the benefit of U.S. Provisional Patent Application Ser. No. 61/834,219 filed Jun. 12, 2013, entitled “Adjusting A Transparent Display With An Image Capturing Device,” the entire disclosure of the application being considered part of the disclosure of this application and hereby incorporated by reference.

### BACKGROUND

Transparent displays, such as a transparent light emitting display (LED), may be provided to augment pre-existing display units. For example, mechanical gauges in a vehicle may be presented along with a transparent display to emphasize or supplant the information provided by the mechanical gauges.

Multiple transparent displays may be provided to further augment an existing display. In addition to providing the multiple transparent displays along with mechanical displays, the multiple transparent displays may be provided as a stand-alone unit.

The multiple transparent displays, when superimposed upon each other, may provide a three-dimensional (3D) effect. In particular, an image may be provided on a first layer and altered slightly on a second layer to produce a combined image. The combined image may appear to the viewer as 3D. Multiple transparent displays may be referred to as multi-layer transparent displays throughout this disclosure.

Thus, by providing the viewer with a 3D image, a multiple transparent display may achieve a more graphically stimulating experience that a mere two-dimensional (2D) graphical presentation. The 3D combined image may be more robust in alerting the viewer with information associated with the multiple transparent displays.

In certain applications, such as a dashboard display of a vehicle, presenting 31) multi-layer transparent displays may lead to an enhanced user experience. For example, the 3D multi-layer transparent displays may be placed over a mechanical gauge integrated as part of the vehicle. The 3D multi-layer transparent display may cause the mechanical gauge to appear as 3D. This 3D appearance may serve as an enhanced user experience.

When 3D multi-layer transparent displays are used, a parallax effect may also be produced. Parallax is a displacement or difference in the apparent position of an object viewed along two different lines of sight, and is measured by the angle or semi-angle of inclination between those two lines.

The introduction of parallax, in the context of certain displays, may be considered as disruptive to a viewer. For example, dashboards of a vehicle that use a needle-style speedometer gauge may experience a parallax effect when viewed at different angles. When the dashboard is viewed from directly in front, the speed may show exactly 60; but when viewed from the passenger seat a needle associated with the mechanical gauge may appear to show a slightly different or misaligned graphic, due to the angle of viewing.

### SUMMARY

A system and a method for adjusting a transparent display with an image capturing device is provided. The system includes a command module to instigate a monitoring of the multi-layer transparent display with the image capturing device; an image receiving module to receive a first image from the image capturing device and a second image from the image capturing device after a predetermined interval; an image processing module to process the received first image and the second image; and a display driving module to re-render the multi-layer transparent display based on the processing of the received first image and the second image.

### DESCRIPTION OF THE DRAWINGS

The detailed description refers to the following drawings, in which like numerals refer to like items, and in which:

FIG. 1 is a block diagram illustrating an example computer.

FIG. 2 illustrates an example of a system for adjusting a multi-layer transparent display.

FIG. 3 illustrates an example of a method for adjusting a multi-layer transparent display.

FIGS. 4(A) and 4(B) illustrate an example implementation of system depicted in FIG. 2.

FIGS. 5(A) and 5(B) illustrate an example implementation of system depicted in FIG. 2.

### DETAILED DESCRIPTION

Multi-layer transparent displays may be used to create a 3D effect. The multi-layer transparent displays may be composed of layers of any sort of transparent display, such a transparent LCD panels, transparent OLEDs, for example. The essence of a transparent display is that a viewer may be able to see the contents of the other side of the display, while viewing light emitting diodes on the display itself. The multi-layer transparent displays may be used in conjunction with other static elements, like a mechanical gauge of a vehicle.

For example, the multi-layer transparent display may be placed in front of the mechanical gauge, and situated in between an operator of a vehicle and the mechanical gauge. The multi-layer transparent display may be configured to be illuminated in a way to augment the mechanical gauge.

In a specific example, the multi-layer transparent display may illuminate in a fashion to provide a 3D display. The 3D display may serve to provide an enhanced user experience versus a conventional 2D display.

However, in the presentation of a 3D display, parallax effects are introduced. The parallax effect may be realized by the operator of the vehicle in response to the operator moving their head or body from one position to another. In a certain situation, due to the introduced parallax effect, information provided from the mechanical gauges may not be accurately conveyed to the operator.

Disclosed herein are systems and methods for reducing parallax with displays. In several of the examples discussed below, a multi-layer transparent display is employed. However, the aspects disclosed herein may be applied to single layer transparent displays as well.

The systems and methods disclosed herein employ an image capturing device that is placed behind a transparent display. Thus, due to the transparent nature of the multi-layer transparent display, the image capturing device may accurately detect a viewing experience of the multi-layer transparent display.

In particular, the image capturing device may detect that a viewer's head position has moved. Accordingly, the multi-layer transparent display may adjust a presentation to compensate for this movement, and thus, avoid the effects of parallax.

Further, the image capturing device may detect that an object is obstructing the transparent display. For example, if the multi-layer transparent display is situated in a vehicle, and an object in the vehicle, such as a steering wheel, obstructs the multi-layer transparent display, the methods and systems disclosed herein may adaptively change the presentation on the multi-layer transparent display to compensate for the obstruction by the steering wheel.

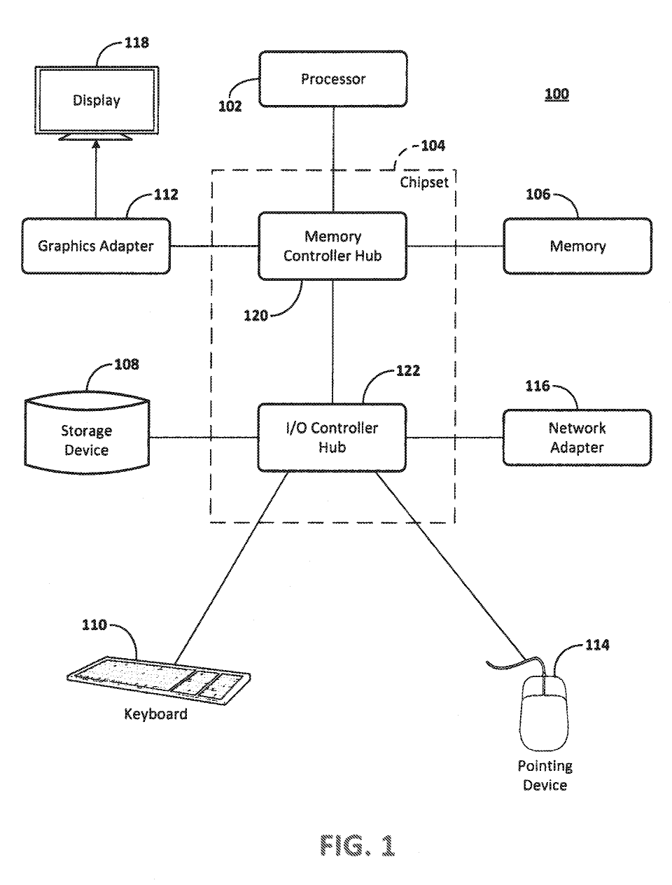

FIG. 1 is a block diagram illustrating an example computer 100. The computer 100 includes at least one processor 102 coupled to a chipset 104. The chipset 104 includes a memory controller hub 120 and an input/output (I/O) controller hub 122. A memory 106 and a graphics adapter 112 are coupled to the memory controller hub 120, and a display 118 is coupled to the graphics adapter 112. A storage device 108, keyboard 110, pointing device 114, and network adapter 116 are coupled to the I/O controller hub 122. Other embodiments of the computer 100 may have different architectures.

The storage device 108 is a non-transitory computer-readable storage medium such as a hard drive, compact disk read-only memory (CD-ROM), DVD, or a solid-state memory device. The memory 106 holds instructions and data used by the processor 102. The pointing device 114 is a mouse, track ball, or other type of pointing device, and is used in combination with the keyboard 110 to input data into the computer system 100. The graphics adapter 112 displays images and other information on the display 118. The network adapter 116 couples the computer system 100 to one or more computer networks.

The computer 100 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 108, loaded into the memory 106, and executed by the processor 102.

The types of computers used by the entities and processes disclosed herein can vary depending upon the embodiment and the processing power required by the entity. The computer 100 may be a mobile device, tablet, smartphone or any sort of computing element with the above-listed elements. For example, a video corpus, such as a hard disk, solid state memory or storage device, might be stored in a distributed database system comprising multiple blade servers working together to provide the functionality described herein. The computers can lack some of the components described above, such as keyboards 110, graphics adapters 112, and displays 118.

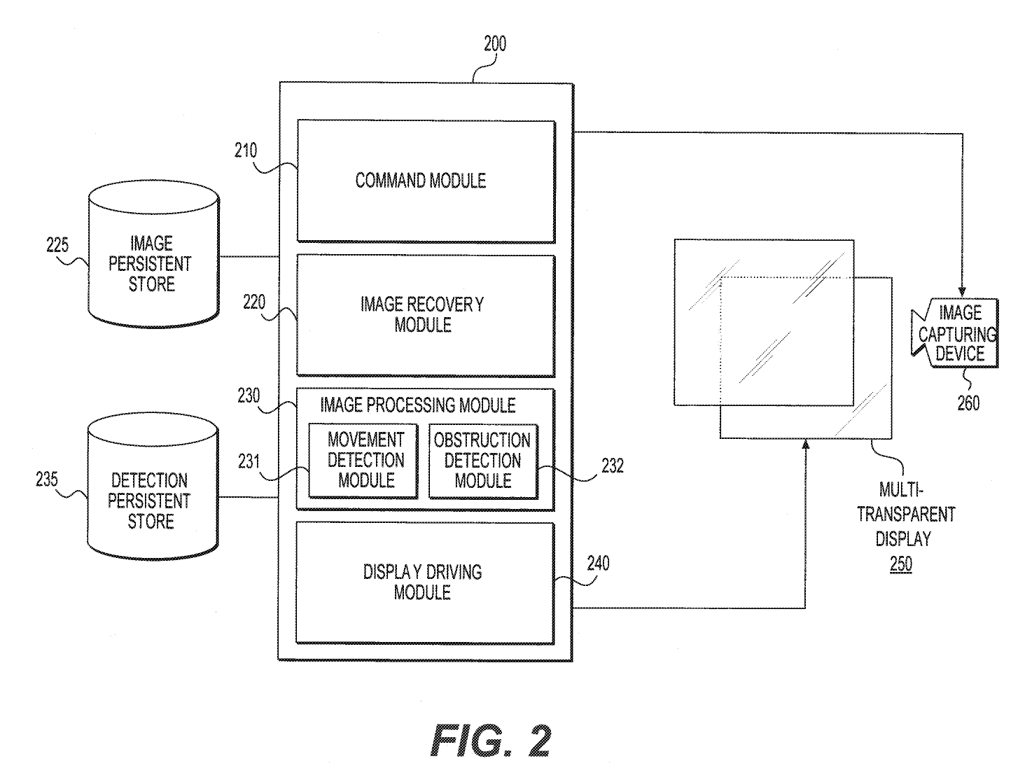

FIG. 2 illustrates an example of a system 200 for adjusting a multi-layer transparent display 250. The system 200 may be incorporated as a device, such as computer 100. The system 200 includes a command module 210, an image receiving module 220, an image processing module 230, and a display driving module 240. The image receiving module 220 may be implemented to communicate with an image capturing device 260. Alternatively, the system 200 may be incorporated with the image capturing device 260.

The command module 210 receives an indication to instigate the system 200. The system 200 may be initiated through various ways, such as by a user indication, automatically engaged in response to the multi-layer transparent display 250 being turned on, for example. In this way, the aspects associated with system 200 may be selectively controlled to be initiated or not based on a user preference or system configuration.

The image receiving module 220, in response the command module 210 receiving an indication to perform the adjustment of system 200, captures an image in the surrounding area in front of a multi-layer transparent display 250. The image receiving module 220 may initiate the image capturing device 260 to capture an image at a predetermined interval. The image capturing device 260 may be positioned behind the multi-layer transparent display 250 in a side opposing a side in which a viewer is oriented relative to the multi-layer transparent display 250.

The image capturing device 260 may be positioned and oriented in a manner to capture the surrounding area associated with the viewer. The image capturing device 260 may be adjustable, thereby providing the viewer the ability to move the image capturing device 260 to ensure that the viewer is captured while observing the multi-layer transparent display 250.

The image receiving module 220 may store the image in an image persistent store 225. The image persistent store may be any sort of storage device, such as storage device 108 discussed above.

The image processing module 230 processes the image received by the image receiving module 220, and performs signal processing to identify the image stored by the image receiving module 220. The image processing module 230 includes a movement detection module 231 and an obstruction detection module 232.

The movement detection module 231 compares a latest version of the image stored in the image persistent store 225 with a previous version, and detects if an object has moved or been displaced. For example, the movement detection module 231 may identify a feature associated with a face, such as a viewer's eyes, and detect if the eye location has changed. If an identified object has changed, the movement detection module 231 may store the detected changed location in the detection persistent store 235.

The obstruction detection module 232 may determine if an object is in between a viewer and the multi-layer transparent display 250. For example, if a steering wheel or some other object is detected as being between the multi-layer transparent display 250 and the viewer, the detection of this object also may be recorded in the detection persistent store 235.

The detection performed by the movement detection module 231 and the obstruction detection module 232 may be performed via a signal processing technique.

The display driving module 240 may modify the presentation of the multi-layer transparent display 250 based on the data stored in the detection persistent store 235.

If the multi-layer transparent display 250 is presently displaying a 3D object, the 3D object may be adjusted based on a detection that the viewer's eyes has moved to a new position. One such adjustment is to reduce the effects of parallax. The 3D object may be re-rendered to avoid the effect of parallax based on the eyes being in a new location.

In another example, the multi-layer transparent display 250 may re-render a display so that a detected object obstructing a viewer does not cause information associated with the multi-layer transparent display 250 to not be visible to the viewer. For example, if the obstruction detection module 232 determines that an object is in between the viewer and the multi-layer transparent display 250, the display driving module 240 may send a signal to the multi-layer transparent display 250 to re-render a display so that viewer of the multi-layer transparent display 250 is capable of viewing the object.

The display driving module 240 may be equipped with a signal processing engine capable of interfacing with various multi-layer transparent displays. The aspects disclosed in relation to system 200 may be performed at a granularity predetermined by the viewer or an implementer of system 200. Thus, the refresh rate at which images are captured, processed and subsequently used to modify a displayed object on a multi-layer transparent display 250 may be predetermined.

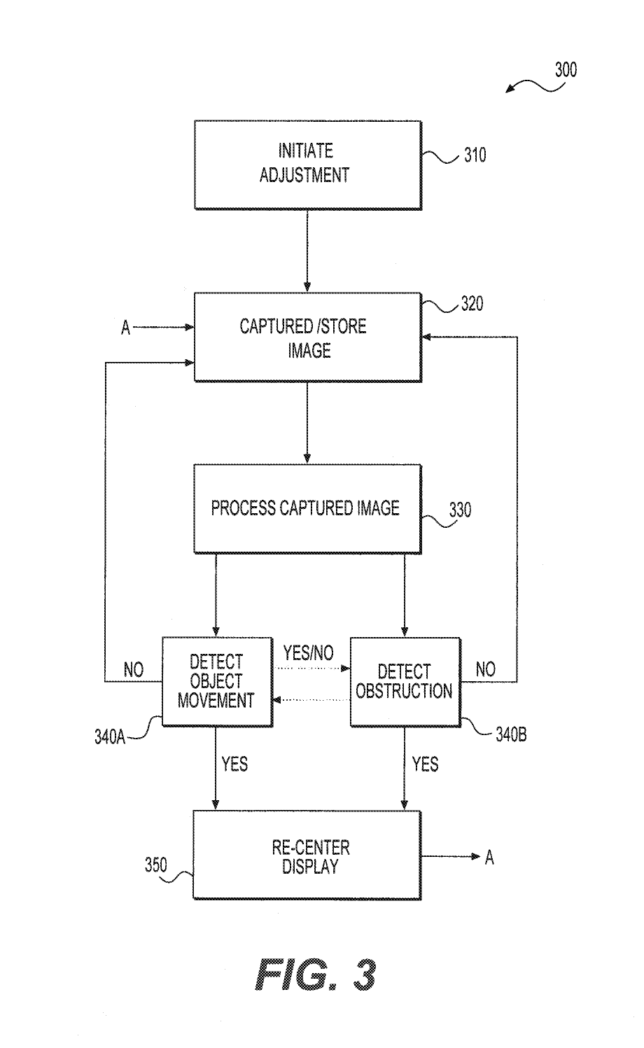

FIG. 3 illustrates an example of a method 300 for adjusting a transparent display with an image capturing device 260. The method 300 may be implemented on the system 200 depicted in FIG. 2.

In operation 310, an indication to initiate adjustments based on the aspects associated with method 300 is received. The indication may be sourced from a user in the area of the multi-layer transparent display 250, or alternatively, through an automatic process based on a stimulus associated with an implementation of the multi-layer transparent display 250. For example, if the multi-layer transparent display is implemented in a vehicle, the stimulus may be defined as an operator instigating the start of the vehicle.

In operation 320, an image capturing device 260 placed on a side opposing a viewer of the multi-layer transparent display 250, captures and stores an image. The image capturing device 260 may be placed in an orientation to effectively capture the viewer observing the multi-layer transparent display 250.

In operation 330, the image captured in operation 320 is processed. The image may undergo various digital signal processing techniques to identify objects, such as facial features. Further, the image may be compared against a previous image to detect changes. Depending on the implementation of method 300, the method 300 may proceed to operation 340A, operation 340B, or both.

In operation 340A, employing the image processing of operation 330, a determination is made as to whether an identified object moved. For example, if the identified object is a facial feature (such as a viewer's eyes), in operation 340A, a recordation of the identified object moving is made. In addition to detecting movement, an amount of movement may also be recorded.

In operation 340B, an obstruction is detected. The obstruction may be defined as any object that prevents a viewer from observing the contents of the multi-layer transparent display 250. For example, if the multi-layer transparent display 250 is implemented in a vehicle, behind a steering wheel, the obstruction may be caused by the steering wheel being moved to a different location. The obstruction may be a whole blocking, or a partial blocking of the multi-layer transparent display 250.

In operation 350, based on the detections of operations 340A and/or 340B, the display is re-rendered. For example, if a facial feature is detected to move, the image may be re-rendered to compensate for parallax effects (i.e. reducing parallax effects). In another example, if an obstruction is detected, the image may be moved so that the image is no longer obstructed on the multi-layer transparent display 250.

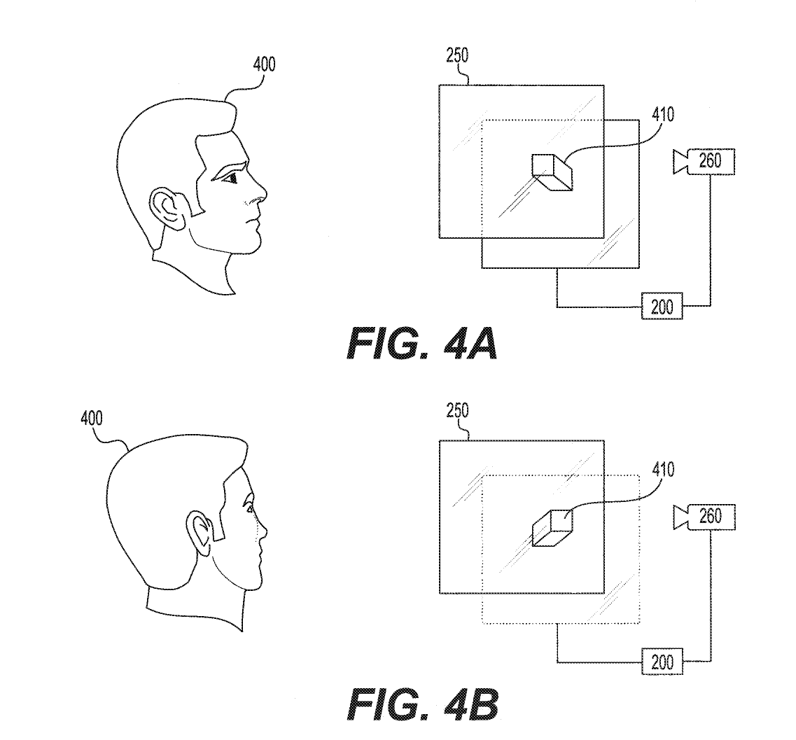

FIGS. 4(A) and 4(B) illustrate an example implementation of system 200. Referring to FIGS. 4(A) and 4(B), a viewer 400 is observing an object 410 being displayed on a multi-layer transparent display 250. The multi-layer transparent display 250 is implemented along with the system 200 and an image capturing device 260.

In FIG. 4(A), the viewer 400 is observing an object 410. The object 410 may be rendered in a 3D fashion. Alternatively, the object 410 may appear as 3D when superimposed over a fixed display, such as a mechanical gauge of a vehicle. The image capturing device 260 captures images of the viewer 400 at a predetermined time interval.

In FIG. 4(B), the viewer 400 turns his head. The image capturing device 260 captures the viewer 400's head, and processes that the viewer 400's eyes are displaced. Based on this displacement, the object 410 is re-rendered. The re-rendering may be performed to negate any parallax effect associated with the viewer 400's head movement. Thus, the system 200 may employ an algorithm or technique for re-rendering the object 410 based on the amount of displacement associated with the viewer 400.

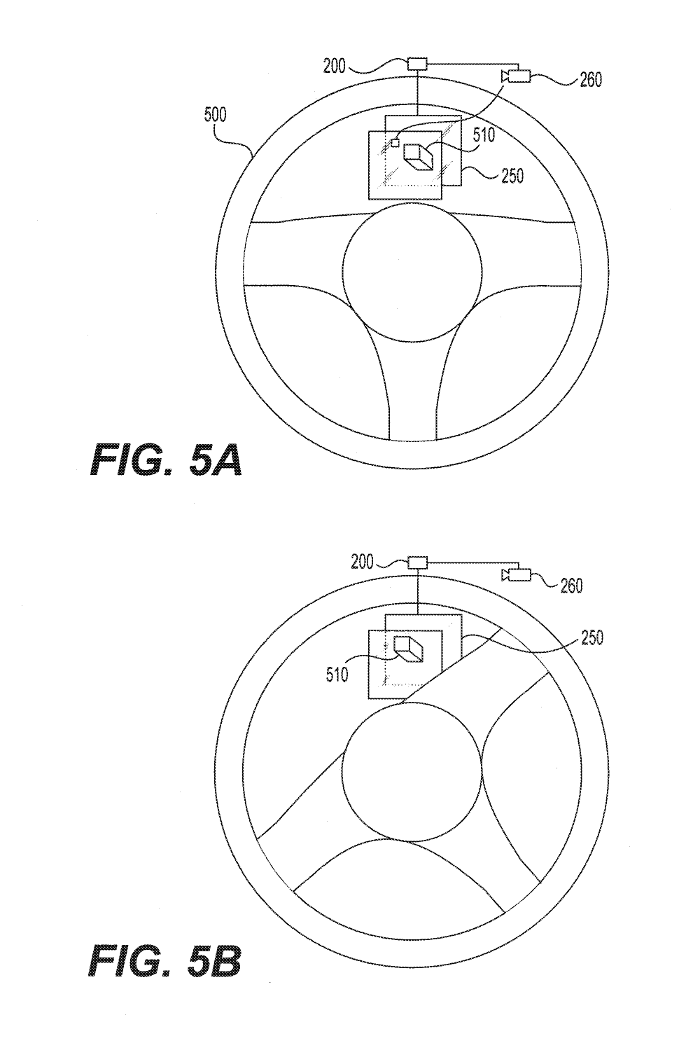

FIGS. 5(A) and 5(B) illustrate an example implementation of system 200 in a vehicle. Referring to FIGS. 5(A) and 5(B), a multi-layer transparent display 250 is implemented behind a steering wheel 500. An image capturing device 260 is placed behind the multi-layer transparent display 250, at a surface opposite the surface being viewed of the multi-layer transparent display 250.

Referring to FIG. 5(A), the steering wheel 500 is at a first position. In this position, the multi-layer transparent display 250 is not being obstructed. The multi-layer transparent display 250 displays object 510, and the viewer of the multi-layer transparent display 250 may be able to view the object 510 in an un-obstructed manner.

Referring to FIG. 5(B), the steering wheel 500 is displaced to a second position. The second position causes the multi-layer transparent display 500 to be partially obstructed by the steering wheel 500. The image capturing device 260 captures an image evidencing this obstruction, and through the aspects described above in conjunction with system 200, the object 510 is re-rendered on the multi-layer transparent display 250 to be located on a portion of the multi-layer transparent display 250 not obstructed by the steering wheel 500.

Certain of the devices shown in FIG. 1 include a computing system. The computing system includes a processor (CPU) and a system bus that couples various system components including a system memory such as read only memory (ROM) and random access memory (RAM), to the processor. Other system memory may be available for use as well. The computing system may include more than one processor or a group or cluster of computing system networked together to provide greater processing capability. The system bus may be any of several types of bus structures including a memory bus or memory controller, a peripheral bus, and a local bus using any of a variety of bus architectures. A basic input/output (BIOS) stored in the ROM or the like, may provide basic routines that help to transfer information between elements within the computing system, such as during start-up. The computing system further includes data stores, which maintain a database according to known database management systems. The data stores may be embodied in many forms, such as a hard disk drive, a magnetic disk drive, an optical disk drive, tape drive, or another type of computer readable media which can store data that are accessible by the processor, such as magnetic cassettes, flash memory cards, digital versatile disks, cartridges, random access memories (RAMs) and, read only memory (ROM). The data stores may be connected to the system bus by a drive interface. The data stores provide nonvolatile storage of computer readable instructions, data structures, program modules and other data for the computing system.

To enable human (and in some instances, machine) user interaction, the computing system may include an input device, such as a microphone for speech and audio, a touch sensitive screen for gesture or graphical input, keyboard, mouse, motion input, and so forth. An output device can include one or more of a number of output mechanisms. In some instances, multimodal systems enable a user to provide multiple types of input to communicate with the computing system. A communications interface generally enables the computing device system to communicate with one or more other computing devices using various communication and network protocols.

The preceding disclosure refers to a number of flow charts and accompanying descriptions to illustrate the embodiments represented in FIG. 3. The disclosed devices, components, and systems contemplate using or implementing any suitable technique for performing the steps illustrated in these figures. Thus, FIG. 3 is for illustration purposes only and the described or similar steps may be performed at any appropriate time, including concurrently, individually, or in combination. In addition, many of the steps in these flow charts may take place simultaneously and/or in different orders than as shown and described. Moreover, the disclosed systems may use processes and methods with additional, fewer, and/or different steps.

Embodiments disclosed herein can be implemented in digital electronic circuitry, or in computer software, firmware, or hardware, including the herein disclosed structures and their equivalents. Some embodiments can be implemented as one or more computer programs, i.e., one or more modules of computer program instructions, encoded on a tangible computer storage medium for execution by one or more processors. A computer storage medium can be, or can be included in, a computer-readable storage device, a computer-readable storage substrate, or a random or serial access memory. The computer storage medium can also be, or can be included in, one or more separate tangible components or media such as multiple CDs, disks, or other storage devices. The computer storage medium does not include a transitory signal.

As used herein, the term processor encompasses all kinds of apparatus, devices, and machines for processing data, including by way of example a programmable processor, a computer, a system on a chip, or multiple ones, or combinations, of the foregoing. The processor can include special purpose logic circuitry, e.g., an FPGA (field programmable gate array) or an ASIC (application-specific integrated circuit). The processor also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, module, engine, software, software application, script, or code) can be written in any form of programming language, including compiled or interpreted languages, declarative or procedural languages, and the program can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

To provide for interaction with an individual, the herein disclosed embodiments can be implemented using an interactive display, such as a graphical user interface (GUI). Such GUI's may include interactive features such as pop-up or pull-down menus or lists, selection tabs, scannable features, and other features that can receive human inputs.

The computing system disclosed herein can include clients and servers. A client and server are generally remote from each other and typically interact through a communications network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

We claim:

1. A system for adjusting a multi-layer transparent display with an image capturing device, comprising:
a data store comprising a computer readable medium storing a program of instructions for the adjusting of the multi-layer transparent display;
a processor that executes the program of instructions;
a command module to instigate a monitoring of the multi-layer transparent display with the image capturing device;
an image receiving module to receive a first image from the image capturing device and a second image from the image capturing device after a predetermined interval;
an image processing module to process the received first image and the second image; and
a display driving module to re-render the multi-layer transparent display based on the processing of the received first image and the second image.

2. The system according to claim 1, wherein the image processing module further comprises a movement detection module to detect that an identified object of the first image and the second image is displaced.

3. The system according to claim 2, wherein the display driving module re-renders the multi-layer transparent display based on the detected displacement.

4. The system according to claim 3, wherein the re-rendering is performed to correct a parallax effect based on the detected displacement.

5. The system according to claim 1, wherein the image processing module further comprises an obstruction detection module to detect that an object of either the first or second image obstructs an item rendered in the multi-layer transparent display.

6. The system according to claim 5, wherein the display driving module re-renders the item based on the detected object.

7. The system according to claim 1, wherein the multi-layer transparent display is implemented along with a dashboard of a vehicle.

8. The system according to claim 1, wherein the image processing module further comprises:
a movement detection module to detect that an identified object of the first image and the second image is displaced; and
an obstruction detection module to detect that an object of either the first or second image obstructs an item rendered in the multi-layer transparent display.

9. The system according to claim 1, wherein the multi-layer transparent display is a transparent organic light emitting display (OLED).

10. A method performed by a processor for adjusting a multi-layer transparent display with an image capturing device, comprising:
initiating a monitoring of the multi-layer transparent display via the image capturing device;
capturing a first image from the image capturing device, and a second image from the image capturing device after a predetermined interval;
processing the received first image and the second image; and
re-rendering the multi-layer transparent display based on the processing of the received first image and the second image,
wherein at least of the initiating, capturing, processing and re-rendering is performed by the processor.

11. The method according to claim 10, wherein the processing further comprises detecting that an identified object of the first image and the second image is displaced.

12. The method according to claim 11, wherein the re-rendering of the multi-layer transparent display is based on the detected displacement of the identified object.

13. The method according to claim 12, wherein the re-rendering is performed to correct a parallax effect based on the detected displacement.

14. The method according to claim 10, wherein the processing further comprises detecting that an object of either the first or second image obstructs an item rendered in the multi-layer transparent display.

15. The method according to claim 14, wherein the re-rendering of the item is based on the detected object.

16. The method according to claim 10, wherein the multi-layer transparent display is implemented along with a dashboard of a vehicle.

17. The method according to claim 10, wherein the processing further comprises:
detecting that an identified object of the first image and the second image is displaced; and
detecting that an object of either first or second image obstructs an item rendered in the multi-layer transparent display.

18. The method according to claim 10, wherein the multi-layer transparent display is a transparent organic light emitting display (OLED).

19. A system for adjusting a transparent display with an image capturing device, comprising:
a data store comprising a computer readable medium storing a program of instructions for the adjusting of the multi-layer transparent display;
a processor that executes the program of instructions;
a command module to instigate a monitoring of the multi-layer transparent display with the image capturing device;
an image receiving module to receive a first image from the image capturing device and a second image from the image capturing device after a predetermined interval;
an image processing module to process the received first image and the second image; and
a display driving module to re-render the multi-layer transparent display based on the processing of the received first image and the second image.

20. The system according to claim 19, wherein the image processing module further comprises:
a movement detection module to detect that an identified object of the first image and the second image is displaced; and
an obstruction detection module to detect that an object of either the first or second image obstructs an item rendered in the transparent display.

21. The system according to claim 1, wherein the transparent display overlays a mechanical display.
